(19) United States
(12) Patent Application Publication
(10) Pub. No.: US 2007/0117776 A1
(43) Pub. Date: May 24, 2007

(54) Low Dose Therapy Of DNA Methylation Inhibitors

(76) Inventor: John Lyons, London (GB)

Correspondence Address: WILSON SONSINI GOODRICH & ROSATI, 650 PAGE MILL ROAD, PALO ALTO, CA, 94304-1050, US

(21) Appl. No.: 11/556,061

(22) Filed: Nov. 2, 2006

Related U.S. Application Data
(63) Continuation of application No. 60/733,408, filed on Nov. 4, 2005.

(51) Int. Cl. A61K 31/7072

(52) U.S. Cl. 514/49

(57) ABSTRACT

Methods are provided for treating patients with hematological disorders such as acute myeloid leukemia (AML), chronic myelogenous leukemia (CML), and the myelodysplastic syndromes (MDS). By administering a DNA methylation inhibitor to the patients following unique dosing regimens, the diseases can be efficaciously treated with reduced toxic side effects.

### CROSS-REFERENCE

This application claims the benefit of U.S. Provisional Application No. 60/733,408, filed Nov. 4, 2005, which is incorporated herein by reference in its entirety.

### BACKGROUND OF THE INVENTION

1. Field of the Invention

The present invention relates to compounds and compositions of DNA methylation inhibitors such as decitabine (or 5-aza-2′-deoxycytidine) and azacitidine (or 5-azacytidine), and methods for formulating and administering these compounds or compositions to a subject in need thereof.

2. Description of Related Art

A few nucleosides of azacytosine, such as decitabine and azacitidine have been developed as antagonists of their related natural nucleosides, cytidine and 2′-deoxycytidine, respectively. The only structural difference between azacytosine and cytosine is the presence of a nitrogen atom at position 5 of the cytosine ring in azacytosine as compared to a carbon at this position for cytosine.

Two isomeric forms of decitabine can be distinguished. The β-anomer is the active form. The modes of decomposition of decitabine in aqueous solution are (a) conversion of the active β-anomer to the inactive α-anomer (Pompon et al. (1987) J. Chromat. 388:113-122); (b) ring cleavage of the aza-pyrimidine ring to form N-(formylamidino)-N′-β-D-2′-deoxy-(ribofuranosy)-urea (Mojaverian and Repta (1984) J. Pharm. Pharmacol. 36:728-733); and (c) subsequent forming of guanidine compounds (Kissinger and Stemm (1986) J. Chromat. 353:309-318).

Decitabine (5-Aza-2-deoxycytidine; 4-amino-1-(2-deoxy-β-D-erythro-pentofuranosyl)-1,3,5-triazin-2(1H)-one) possesses multiple pharmacological characteristics. At a molecular level, it is S-phase dependent for incorporation into DNA. At a cellular level, decitabine can induce cell differentiation and exert hematological toxicity. Despite having a short half life in vivo, decitabine has an excellent tissue distribution.

One of the functions of decitabine is its ability to specifically and potently inhibit DNA methylation. Methylation of cytosine to 5-methylcytosine occurs at the level of DNA. Inside the cell, decitabine is first converted into its active form, the phosphorylated 5-aza-deoxycytidine, by deoxycytidine kinase which is primarily synthesized during the S phase of the cell cycle. The affinity of decitabine for the catalytical site of deoxycytidine kinase is similar to the natural substrate, deoxycytidine. After conversion to its triphosphate form by deoxycytidine monophosphate kinase and nucleoside diphosphokinase, decitabine is incorporated into replicating DNA at a rate similar to that of the natural substrate, dCTP. Bouchard and Momparler (1983) Mol. Pharmacol. 24:109-114.

Incorporation of decitabine into the DNA strand has a hypomethylation effect. Each class of differentiated cells has its own distinct methylation pattern. After chromosomal duplication, in order to conserve this pattern of methylation, the 5-methylcytosine on the parental strand serves to direct methylation on the complementary daughter DNA strand. Substituting the carbon at the 5 position of the cytosine for a nitrogen interferes with this normal process of DNA methylation. The replacement of 5-methylcytosine with decitabine at a specific site of methylation produces an irreversible inactivation of DNA methyltransferase, presumably due to formation of a covalent bond between the enzyme and decitabine. Juttermann et al. (1994) Proc. Natl. Acad. Sci. USA 91:11797-11801. By specifically inhibiting DNA methyltransferase, the enzyme required for methylation, the aberrant methylation of the tumor suppressor genes can be prevented.

Decitabine is commonly supplied as a sterile lyophilized powder for injection, together with buffering salt, such as potassium dihydrogen phosphate, and pH modifier, such as sodium hydroxide. For example, decitabine is supplied by SuperGen, Inc., as lyophilized powder packed in 20 mL glass vials, containing 50 mg of decitabine, monobasic potassium dihydrogen phosphate, and sodium hydroxide. When reconstituted with 10 mL of sterile water for injection, each mL contains 5 mg of decitabine, 6.8 mg of KH₂PO₄, and approximately 1.1 mg NaOH. The pH of the resulting solution is 6.5-7.5. The reconstituted solution can be further diluted to a concentration of 1.0 or 0.1 mg/mL in cold infusion fluids, i.e., 0.9% Sodium Chloride; or 5% Dextrose; or 5% Glucose; or Lactated Ringer's. The unopened vials are typically stored under refrigeration (2-8° C.; 36-46° F.), in the original package.

Decitabine is most typically administrated to patients by injection, such as by a bolus I.V. injection, subcutaneous injection, continuous I.V. infusion, or I.V. infusion. The length of I.V. infusion is limited by the decomposition of decitabine in aqueous solutions. Similar to decitabine, azacitidine also suffers from the same chemical instability in aqueous solutions. Azacitidine has been approved by the FDA for treating myelodysplastic syndrome (MDS) subtypes by administrating to the patients at 75 mg/m² subcutaneously daily for seven days every four weeks.

Thus, to effectively treat epigenetic diseases associated aberrant DNA methylation such as MDS, sickle cell anemia, thalassemia and cancer, there exists a need for innovative dosing regimens for administering a DNA methylation inhibitor such as decitabine and azacitidine to patients by balancing multiple factors in the clinic: the chemical stability, therapeutic efficacy, and adverse side effects of the drug, ease of administration, patient compliance and comfort, and maximized drug usage. The present invention provides such innovative methods for treating patients having an epigenetic disease.

### SUMMARY OF THE INVENTION

The present invention provides optimized dosing regimens for a DNA methylation inhibitor for the treatment of patients having, or being at risk for, epigenetic diseases or conditions, such as hematological disorders and cancer.

In a preferred embodiment, a low dose of the DNA methylation inhibitor (e.g., decitabine and azacytidine) is administered to the patient, ranging from about 0.1 to about 50 mg/m², preferably from about 1 to 40 mg/m², more preferably from about 5 to 30 mg/m², still more preferably from about 1 to 25 mg/m², or from about 5 to about 25 mg/m², most preferably about 10 to about 20 mg/m².

In particular embodiments, the DNA methylation inhibitor is decitabine and is administered to the patient at a dose of about 5, 6, 7, 8, 9, 10, 11, 12, 13, 14, 15, 16, 17 18, 19, 20, 21, 22, 23, 24, or 25 mg/m² once, twice, thrice, four times, five times, or six times a day depending the patient's individual needs and the treatment benefit to be achieved. Optionally, the DNA methylation inhibitor is administered via intravenous infusion for 1-24 hr, 1-12 hr, 1-8 hr, 1-6 hr, 1-3 hr, 2 hrs, 1.5 hr, 1 hr or 0.5 hr.

Such low doses of the DNA methylation inhibitor may be administered with any acceptable route, e.g., orally, parenterally, intraperitoneally, intravenously, intraarterially, transdermally, sublingually, intramuscularly, rectally, transbuccally, intranasally, liposomally, via inhalation, vaginally, intraoccularly, via local delivery (for example by a catheter or stent), subcutaneously, intraadiposally, intraarticularly, or intrathecally. Preferably the DNA methylation inhibitor is administered intravenously or subcutaneously.

Also provided are variations of the above-described dosing regimens, formulations of the DNA methylation inhibitor, combination therapy with other therapeutic agents, and indications that may be effectively treated via the inventive dosing schedules disclosed herein.

### DETAILED DESCRIPTION OF THE INVENTION

The present invention provides innovative methods for treating patients with epigenetic diseases, especially those associated with aberrant DNA methylation. By administering a DNA methylation inhibitor to the patients following unique dosing regimens, desirable clinical outcomes are achievable.

Recently it has been discovered that epigenetic changes, especially DNA methylation, play important roles in gene regulation, cell differentiation and disease progression. DNA methylation is a commonly occurring modification of human DNA. It involves the addition of a methyl group to cytosine residues at CpG dinucleotides, a reaction that is catalyzed by DNA methyltransferase (DNMT) enzymes. CpG dinucleotides are gathered in clusters called CpG islands, which are unequally distributed across the human genome. There are approximately 30,000 CpG islands in the genome and 50-60% of these are found within the promoter region of genes. CpG islands are primarily unmethylated in normal tissues. As will be described in detail below, the aberrant methylation of CpG islands is clearly related with disease, such as hematological disorders and cancer. It has been demonstrated that the abnormal methylation causes transcriptional repression of numerous genes, leading to tumor growth and development.

Studies of DNA methylation in cancer have uncovered new potential targets for the diagnosis, prognosis and ultimately the treatment of human cancer.

As described above, incorporation of decitabine into DNA produces inhibition of the methylation of cytosine at position 5. Decitabine may exert a cytotoxic action leading to cell death, but may also affect cell differentiation by demethylation. In general, inactive genes that are methylated become active on demethylation. Due to the dual activity of decitabine, it has been a major challenge in the treatment of epigenetic diseases that the optimum dosing regimens with desirable in vivo efficacy and minimum toxic side effects are difficult to be determined.

The present invention provides optimized dosing regimens of a DNA methylation inhibitor for the treatment of patients having, or being at risk for, epigenetic diseases or conditions, such as hematological disorders and cancer. In a preferred embodiment, a low dose of the DNA methylation inhibitor (e.g., decitabine and azacytidine) is administered to the patient. “Low-dose,” for purposes of the present invention, means that the therapeutically effective amount of the DNA methylation inhibitor ranges from about 0.1 to about 50 mg/m², preferably from about 1 to 40 mg/m², more preferably from about 5 to 30 mg/m², still more preferably from about 1 to 25 mg/m², or from about 5 to about 25 mg/m², most preferably about 10 to about 20 mg/m². In particular embodiments, the DNA methylation inhibitor is decitabine and is administered to the patient at a dose of about 5, 6, 7, 8, 9, 10, 11, 12, 13, 14, 15, 16, 17 18, 19, 20, 21, 22, 23, 24, or 25 mg/m² once, twice, thrice, four times, five times, or six times a day, depending on the patient's individual medical needs and the treatment benefit to be achieved. Optionally, the DNA methylation inhibitor is administered via intravenous infusion for 1-24 hr, 1-12 hr, 1-8 hr, 1-6 hr, 1-3 hr, 2 hrs, 1.5 hr, 1 hr or 0.5 hr.

Such low doses of the DNA methylation inhibitor may be administered with any acceptable route, e.g., orally, parenterally, intraperitoneally, intravenously, intraarterially, transdermally, sublingually, intramuscularly, rectally, transbuccally, intranasally, liposomally, via inhalation, vaginally, intraoccularly, via local delivery (for example by a catheter or stent), subcutaneously, intraadiposally, intraarticularly, or intrathecally. Preferably the DNA methylation inhibitor is administered intravenously or subcutaneously.

In a preferred embodiment, the DNA methylation inhibitor is decitabine and is administered to the patient at a dose of about 15 mg/m² via a 3 hr intravenous infusion every 8 hrs for 3 consecutive days as a treatment cycle, resulting in a total amount of about 45 mg/m² decitabine per day, and a total amount of about 135 mg/m² decitabine per treatment cycle. This cycle may be repeated every 3-8 weeks, preferably every 4-6 weeks, and most preferably 6 weeks. The patient may be treated for as long as the patient continues to benefit from such treatment. Accordingly, the present invention contemplates a minimum of 1-2 cycles, preferably 5-15 cycles, and more preferably 7-10 cycles. As will be appreciated by those skilled in the art, several treatment cycles may be required before a treatment benefit becomes apparent. Depending on the magnitude and nature of the treatment benefit, cycles may be discontinued once a treatment benefit becomes fully established, or may be continued, optionally at a reduced dose and/or frequency, to maintain the clinical benefit. The patient preferably is one having a hematological disorder such as a myelodysplastic syndrome (MDS), acute myeloid leukemia (AML) and chronic myeloid leukemia (CML), or tumors.

Based on hematology laboratory values of the patient, or other indicia of treatment tolerability, the dose of decitabine may be adjusted to be lower than 15 mg/m². For patients in need of a reduced dose, preferable exemplary doses range from about 12 to about 6 mg/m², more preferably from about 12 to about 8 mg/m², and most preferably are about 11 mg/m². Myelosuppression resulting in, e.g., reduced white blood cell and platelet counts in peripheral blood are a well-known side-effect of decitabine treatment. Accordingly, it is preferable to allow for sufficient hematological recovery time between decitabine treatment cycles. Non-limiting examples of adequate recovery times between treatment cycles are about 3-about 10 weeks, preferably about 4-about 8 weeks, and more preferably about 6 weeks. A desirable hematologic recovery may be indicated by a return of peripheral white blood cell counts and/or platelet counts to about pretreatment levels, or an absolute neutrophil count (ANC) equal to or higher than about 1,000/μL, and a platelet count equal to or higher than about 50,000/μL. If hematologic recovery from a previous decitabine treatment cycle requires more than six weeks, then the next cycle of decitabine therapy should be delayed and dosing temporarily reduced by following this algorithm:

If recovery requires more than six, but less than eight weeks, decitabine dosing is delayed for up to two weeks and the dose temporarily is reduced to 11 mg/m² via a 3 hr infusion very 8 hrs for 3 consecutive days (resulting in 33 mg/m²/day, 99 mg/m²/cycle) upon restarting therapy.

If recovery requires more than eight, but less than 10 weeks, the patient should be assessed for disease progression (by bone marrow aspirates); in the absence of progression, the decitabine dose should be delayed up to two more weeks and the dose reduced to 11 mg/m² via a 3 hr infusion very 8 hrs for 3 consecutive days (resulting in 33 mg/m²/day, 99 mg/m²/cycle) upon restarting therapy, then maintained or increased in subsequent cycles as clinically indicated.

If any of the following non-hematologic toxicities are present, decitabine treatment should not be restarted until the toxicity is resolved: 1) serum creatinine≧2 mg/dL; 2) serum glutamic pyruvic transaminase (SGPT), total bilirubin≧2 times upper limit of normal (ULN); and 3) active or uncontrolled infection.

In another preferred embodiment, the DNA methylation inhibitor is decitabine and is administered to the patient at a dose of about 20 mg/m² intravenously over 1 hr per day for 5 consecutive days as a treatment cycle, resulting in a total amount of about 20 mg/m² decitabine per day and a total amount of about 100 mg/m² decitabine per treatment cycle. This cycle may be repeated every 3-8 weeks, preferably every 4-6 weeks, and most preferably 4 weeks. The patient may be treated for as long as the patient continues to benefit from such treatment, for example 2-20 cycles or more, preferably 2-10 cycles, and most preferably 3-6 cycles. As described above, several treatment cycles may be required before a treatment benefit becomes apparent. Depending on the nature and magnitude of the treatment benefit, cycles may be discontinued once a treatment benefit becomes fully established, or may be continued, optionally at a reduced dose and/or frequency, to maintain the clinical benefit. The patient preferably is one having a hematological disorder such as a MDS, acute myeloid leukemia (AML) and chronic myeloid leukemia (CML), or tumors.

Based on hematology laboratory values of the patient, or other indicia of treatment tolerability, the dose of decitabine may be adjusted to be lower than 20 mg/m² For patients in need of such a reduced dose, exemplary doses range from about 12 to about 6 mg/m², more preferably from about 12 to about 8 mg/m², and most preferably are about 10 mg/m² administered intravenously over 1 hr per day for 5 consecutive days. As described above, it is preferable to allow for sufficient hematological recovery time between the decitabine treatment cycles of this embodiment of the invention. Non-limiting examples of adequate recovery times between treatment cycles are about 2 to about 8 weeks, preferably about 3 to about 7 weeks, and more preferably about 4 weeks. A desirable hematologic recovery may be indicated by a return of peripheral white blood cell (e.g. granulocytes) and/or platelet counts to about pretreatment levels (or to about the level preceding the previous treatment cycles), or to an absolute neutrophil count (ANC) equal to or higher than about 1,000/μL, and a platelet count equal to or higher than about 50,000/μL.

In yet another preferred embodiment, the DNA methylation inhibitor is decitabine and is administered to the patient at a dose of about 10 mg/m² intravenously over 1 hr per day for 10 consecutive days or 5 consecutive days a week for 2 weeks as a treatment cycle, resulting in a total amount of about 10 mg/m² decitabine per day and a total amount of about 100 mg/m² decitabine per treatment cycle. As described above, this cycle may be repeated at intervals allowing for adequate hematological recovery from the previous treatment cycle, for example every 3-8 weeks, preferably every 4-6 weeks, and most preferably 4 weeks. The patient may be treated for as long as is needed to establish and/or maintain a clinical benefit, provided that the treatment is otherwise reasonably well tolerated, for example 2-20 cycles or more, preferably 2-10 cycles, and most preferably 3-6 cycles. The patient preferably is one having a hematological disorder such as a MDS, acute myeloid leukemia (AML) and chronic myeloid leukemia (CML), or tumors.

In still another preferred embodiment, the DNA methylation inhibitor is decitabine and is administered to the patient at a dose of about 10 mg/m² via bolus subcutaneous injection twice a day (BID) per day for 5 days as a treatment cycle, resulting in a total amount of about 20 mg/m² decitabine per day and a total amount of about 100 mg/m² decitabine per treatment cycle. This cycle may be repeated at intervals allowing for adequate hematological recovery from the preceding treatment cycle, e.g. every 3-8 weeks, preferably every 4-6 weeks, and most preferably 4 weeks. The patient may be treated for as long as is needed to establish and/or maintain a treatment benefit (provided that the treatment is otherwise reasonably well tolerated), e.g. for 2-20 cycles or more, preferably 2-10 cycles, and most preferably 3-6 cycles. The patient preferably is one having a hematological disorder such as a MDS, acute myeloid leukemia (AML) and chronic myeloid leukemia (CML), or tumors.

Compared to a standard chemotherapy primarily focusing on exerting cytotoxic effects on the patient, the dosing regimens provided in the present invention involve much lower doses of a DNA methylation inhibitor. It is believed that providing a low dose therapy of the DNA methylation inhibitor in this manner may reactivate genes that have been silenced by hypermethylation to exert cancer-suppressing effects without indiscriminatory cell killing, thus minimizing toxicity responses normally associated with the therapy at higher doses or high-bolus administration. Such toxicity responses include, but are not limited to, prolonged and significant myelosuppression and neurological problems. Further, by optimizing the time and doses for delivering the DNA methylation inhibitor depending on the route of administration while balancing factors such as chemical stability, ease of handing of the drug, avoiding drug waste and patient's convenience and comfort, the inventive therapy can be used to efficaciously treat patients having an epigenetic disease without severe toxic side effects. In addition, in the preferred embodiments, the time of continuous intravenous infusion is shortened to be 3 hrs or less, thus avoiding changing infusion bags to preserve the potency of the drug in the infusion fluid at room temperature.

The details of the invention are described further in the sections below.

1. Aberrant Hypermethylation of Cancer-Related Genes

In mammalian cells, approximately 3% to 5% of the cytosine residues in genomic DNA are present as 5-methylcytosine. Ehrlich et al (1982) Nucleic Acid Res. 10:2709-2721. This modification of cytosine takes place after DNA replication and is catalyzed by DNA methyltransferase using S-adenosyl-methionine as the methyl donor. Approximately 70% to 80% of 5-methylcytosine residues are found in the CpG sequence. Bird (1986) Nature 321:209-213. This sequence, when found at a high frequency, in the genome, is referred to as CpG islands. Unmethylated CpG islands are associated with housekeeping genes, while the islands of many tissue-specific genes are methylated, except in the tissue where they are expressed. Yevin and Razin (1993) in DNA Methylation: Molecular Biology and Biological Significance. Basel: Birkhauser Verlag, p 523-568. This methylation of DNA has been proposed to play an important role in the control of expression of different genes in eukaryotic cells during embryonic development. Consistent with this hypothesis, inhibition of DNA methylation has been found to induce differentiation in mammalian cells. Jones and Taylor (1980) Cell 20:85-93.

Methylation of DNA in the regulatory region of a gene can inhibit transcription of the gene. This may be because 5-methylcytosine protrudes into the major groove of the DNA helix, which interferes with the binding of transcription factors.

The methylated cytosine in DNA, 5-methylcytosine, can undergo spontaneous deamination to form thymine at a rate much higher than the deamination of cytosine to uracil. Shen et al. (1994) Nucleic Acid Res. 22:972-976. If the deamination of 5-methylcytosine is unrepaired, it will result in a C to T transition mutation. For example, many “hot spots” of DNA damages in the human p53 gene are associated with CpG to TpG transition mutations. Denissenko et al. (1997) Proc. Natl. Acad. Sci. USA 94:3893-1898.

Other than the p53 gene, many tumor suppressor genes can also be inactivated by aberrant methylation of the CpG islands in their promoter regions. Many tumor-suppressors and other cancer-related genes have been found to be hypermethylated in human cancer cells and primary tumors. Examples of genes that participate in suppressing tumor growth and are silenced by aberrant hypermethylation include, but are not limited to, tumor suppressors such as p15/INK4B (cyclin kinase inhibitor, p16/INK4A (cyclin kinase inhibitor), p73 (p53 homology), ARF/INK4A (regular level p53), Wilms tumor, von Hippel Lindau (VHL), retinoic acid receptor-β (RARβ), estrogen receptor, androgen receptor, mammary-derived growth inhibitor hypermethylated in cancer (HIC1), and retinoblastoma (Rb); Invasion/metastasis suppressor such as E-cadherin, tissue inhibitor metalloproteinase-2 (TIMP-3), mts-1 and CD44; DNA repair/detoxify carcinogens such as methylguanine methyltransferase, hMLH1 (mismatch DNA repair), glutathione S-transferase, and BRCA-1; Angiogenesis inhibitors such as thrombospondin-1 (TSP-1) and TIMP3; and tumor antigens such as MAGE-1.

In particular, silencing of p16 is frequently associated with aberrant methylation in many different types of cancers. The p16/INK4A tumor suppressor gene codes for a constitutively expressed cyclin-dependent kinase inhibitor, which plays a vital role in the control of cell cycle by the cyclin D-Rb pathway. Hamel and Hanley-Hyde (1997) Cancer Invest. 15:143-152. P16 is located on chromosome 9p, a site that frequently undergoes loss of heterozygosity (LOH) in primary lung tumors. In these cancers, it is postulated that the mechanism responsible for the inactivation of the nondeleted allele is aberrant methylation. Indeed, for lung carcinoma cell lines that did not express p16, 48% showed signs of methylation of this gene. Otterson et al. (1995) Oncogene 11:1211-1216. About 26% of primary non-small cell lung tumors showed methylation of p16. Primary tumors of the breast and colon display 31% and 40% methylation of p16, respectively. Herman et al. (1995) Cancer Res. 55:4525-4530.

Aberrant methylation of retinoic acid receptors is also attributed to development of breast cancer, lung cancer, ovarian cancer, etc. Retinoic acid receptors are nuclear transcription factors that bind to retinoic acid responsive elements (RAREs) in DNA to activate gene expression. In particular, the putative tumor suppressor RARβ gene is located at chromosome 3p24, a site that shows frequent loss of heterozygosity in breast cancer. Deng et al. (1996) Science 274:2057-2059. Transfection of RARβcDNA into some tumor cells induced terminal differentiation and reduced their tumorigenicity in nude mice. Caliaro et al. (1994) Int. J. Cancer 56:743-748; and Houle et al. (1993) Proc. Natl. Acad. Sci. USA 90:985-989. Lack of expression of the RAR□ gene has been reported for breast cancer and other types of cancer. Swisshelm et al. (1994) Cell Growth Differ. 5:133-141; and Crowe (1998) Cancer Res. 58:142-148. This reason for lack of expression of RARβ gene is attributed to hypermethylation of RARβ gene. Indeed, methylation of RARβ was detected in 43% of primary colon carcinomas and in 30% of primary breast carcinoma. Cote et al. (1998) Anti-Cancer Drugs 9:743-750; and Bovenzi et al. (1999) Anticancer Drugs 10:471-476.

Hypermethylation of CpG islands in the 5′-region of the estrogen receptor gene has been found in multiple tumor types. Issa et al. (1994) J. Natl. Cancer Inst. 85:1235-1240. The lack of estrogen receptor expression is a common feature of hormone unresponsive breast cancers, even in the absent of gene mutation. Roodi et al. (1995) J. Natl. Cancer Inst. 87:446-451. About 25% of primary breast tumors that were estrogen receptor-negative displayed aberrant methylation at one site within this gene. Breast carcinoma cell lines that do not express the mRNA for the estrogen receptor displayed increased levels of DNA methyltransferase and extensive methylation of the promoter region for this gene. Ottaviano et al. (1994) 54:2552-2555.

Hypermethylation of human mismatch repair gene (hMLH-1) is also found in various tumors. Mismatch repair is used by the cell to increase the fidelity of DNA replication during cellular proliferation. Lack of this activity can result in mutation rates that are much higher than that observed in normal cells. Modrich and Lahue (1996) Annu. Rev. Biochem. 65:101-133. Methylation of the promoter region of the mismatch repair gene (hMLH-1) was shown to correlate with its lack of expression in primary colon tumors, whereas normal adjacent tissue and colon tumors the expressed this gene did not show signs of its methylation. Kane et al. (1997) Cancer Res. 57:808-811.

The molecular mechanisms by which aberrant methylation of DNA takes place during tumorigenesis are not clear. It is possible that the DNA methyltransferase makes mistakes by methylating CpG islands in the nascent strand of DNA without a complementary methylated CpG in the parental strand. It is also possible that aberrant methylation may be due to the removal of CpG binding proteins that “protect” these sites from being methylated. Whatever the mechanism, the frequency of aberrant methylation is a rare event in normal mammalian cells.

Examples of genes that have been found to be aberrantly methylated include, but are not limited to, VHL (the Von Hippon Landau gene involved in renal cell carcinoma); P16/INK4A (involved in lymphoma); E-cadherin (involved in metastasis of breast, thyroid, gastric cancer); hMLH1 (involved in DNA repair in colon, gastric, and endometrial cancer); BRCA1 (involved in DNA repair in breast and ovarian cancer); LKB1 (involved in colon and breast cancer); P15/INK4B (involved in leukemia such as AML and ALL); ER (estrogen receptor, involved in breast, colon cancer and leukemia); 06-MGMT (involved in DNA repair in brain, colon, lung cancer and lymphoma); GST-pi (involved in breast, prostate, and renal cancer); TIMP-3 (tissue metalloprotease, involved in colon, renal, and brain cancer metastasis); DAPK1 (DAP kinase, involved in apoptosis of B-cell lymphoma cells); P73 (involved in apoptosis of lymphomas cells); AR (androgen receptor, involved in prostate cancer); RAR-beta (retinoic acid receptor-beta, involved in prostate cancer); Endothelin-B receptor (involved in prostate cancer); Rb (involved in cell cycle regulation of retinoblastoma); P14ARF (involved in cell cycle regulation); RASSF1 (involved in signal transduction); APC (involved in signal transduction); Caspase-8 (involved in apoptosis); TERT (involved in senescence); TERC (involved in senescence); TMS-1 (involved in apoptosis); SOCS-1 (involved in growth factor response of hepatocarcinoma); PITX2 (hepatocarcinoma breast cancer); MINT1; MINT2; GPR37; SDC4; MYOD1; MDR1; THBS1; PTC1; and pMDR1, as described in Santini et al. (2001) Ann. of Intern. Med. 134:573-586, which is herein incorporated by reference in its entirety.

2. Pharmaceutical Formulations

According to the present invention, the DNA methylation inhibitor (e.g., decitabine and azacitidine) or other therapeutic agents used in conjunction with the DNA methylation inhibitor can be formulated into pharmaceutically acceptable compositions for treating various diseases and conditions

The pharmaceutically-acceptable compositions of the present invention comprise a DNA methylation inhibitor in association with one or more nontoxic, pharmaceutically-acceptable carriers and/or diluents and/or adjuvants and/or excipients, collectively referred to herein as “carrier” materials, and if desired other active ingredients.

The compounds or compositions of the present invention are administered by any route, preferably in the form of a pharmaceutical composition adapted to such a route, as illustrated below and are dependent on the condition being treated.

The compounds and compositions can be, for example, administered orally, parenterally, intraperitoneally, intravenously, intraarterially, transdermally, sublingually, intramuscularly, rectally, transbuccally, intranasally, liposomally, via inhalation, vaginally, intraoccularly, via local delivery (for example by a catheter or stent), subcutaneously, intraadiposally, intraarticularly, or intrathecally.

The pharmaceutical formulation may optionally further include an excipient added in an amount sufficient to enhance the stability of the composition, maintain the product in solution, or prevent side effects (e.g., potential ulceration, vascular irritation or extravasation) associated with the administration of the inventive formulation. Examples of excipients include, but are not limited to, mannitol, sorbitol, lactose, dextrox, cyclodextrin such as, α-, β-, and γ-cyclodextrin, and modified, amorphous cyclodextrin such as hydroxypropyl-, hydroxyethyl-, glucosyl-, maltosyl-, maltotriosyl-, carboxyamidomethyl-, carboxymethyl-, sulfobutylether-, and diethylamino-substituted α-, β-, and γ-cyclodextrin. Cyclodextrins such as Encapsin® from Janssen Pharmaceuticals or equivalent may be used for this purpose.

For oral administration, the pharmaceutical compositions can be in the form of, for example, a tablet, capsule, suspension or liquid. The pharmaceutical composition is preferably made in the form of a dosage unit containing a therapeutically-effective amount of the active ingredient. Examples of such dosage units are tablets and capsules. For therapeutic purposes, the tablets and capsules which can contain, in addition to the active ingredient, conventional carriers such as binding agents, for example, acacia gum, gelatin, polyvinylpyrrolidone, sorbitol, or tragacanth; fillers, for example, calcium phosphate, glycine, lactose, maize-starch, sorbitol, or sucrose; lubricants, for example, magnesium stearate, polyethylene glycol, silica, or talc; disintegrants, for example, potato starch, flavoring or coloring agents, or acceptable wetting agents. Oral liquid preparations generally are in the form of aqueous or oily solutions, suspensions, emulsions, syrups or elixirs may contain conventional additives such as suspending agents, emulsifying agents, non-aqueous agents, preservatives, coloring agents and flavoring agents. Examples of additives for liquid preparations include acacia, almond oil, ethyl alcohol, fractionated coconut oil, gelatin, glucose syrup, glycerin, hydrogenated edible fats, lecithin, methyl cellulose, methyl or propyl para-hydroxybenzoate, propylene glycol, sorbitol, or sorbic acid.

For topical use the compounds in the present invention can also be prepared in suitable forms to be applied to the skin, or mucus membranes of the nose and throat, and can take the form of creams, ointments, liquid sprays or inhalants, lozenges, or throat paints. Such topical formulations further can include chemical compounds such as dimethylsulfoxide (DMSO) to facilitate surface penetration of the active ingredient.

For application to the eyes or ears, the compounds in the present invention can be presented in liquid or semi-liquid form formulated in hydrophobic or hydrophilic bases as ointments, creams, lotions, paints or powders.

For rectal administration the compounds in the present invention can be administered in the form of suppositories admixed with conventional carriers such as cocoa butter, wax or other glyceride.

Alternatively, the compounds in the present invention can be in powder form for reconstitution in the appropriate pharmaceutically acceptable carrier at the time of delivery.

The pharmaceutical compositions can be administered via injection. Formulations for parenteral administration can be in the form of aqueous or non-aqueous isotonic sterile injection solutions (e.g., WFI) or suspensions. These solutions or suspensions can be prepared from sterile powders or granules having one or more of the carriers mentioned for use in the formulations for oral administration. The compounds can be dissolved in polyethylene glycol, propylene glycol, ethanol, corn oil, benzyl alcohol, sodium chloride, and/or various buffers.

In a particular embodiment, decitabine or azacitidine can be formulated into a pharmaceutically acceptable composition comprising the compound solvated in non-aqueous solvent that includes glycerin, propylene glycol, polyethylene glycol, or combinations thereof. It is believed that decitabine or azacitidine will be stable in such pharmaceutical formulations so that the pharmaceutical formulations may be stored for a prolonged period of time prior to use.

In a preferred embodiment, the pharmaceutical formulation contains less than 40% water in the solvent, optionally less than 20% water in the solvent, optionally less than 10% water in the solvent, or optionally less than 1% water in the solvent. In one variation, the pharmaceutical formulation is stored in a substantially anhydrous form. Optionally, a drying agent may be added to the pharmaceutical formulation to absorb water.

Owing to the enhanced stability, the pharmaceutical formulation may be stored and transported at ambient temperature, thereby significantly reducing the cost of handling the drug. Further, the pharmaceutical formulation may be conveniently stored for a long time before being administered to the patient. In addition, the inventive formulation may be diluted with regular infusion fluid (without chilling) and administered to a patient at room temperature, thereby avoiding causing patients' discomfort associated with infusion of cold fluid.

In another embodiment, decitabine or azacitidine is dissolved in glycerin at different concentrations. For example, the formulation may optionally comprise between 0.1 and 200; between 1 and 100; between 1 and 50; between 2 and 50; between 2 and 100; between 5 and 100; between 10 and 100 or between 20 and 100 mg inventive compound per ml of glycerin. Specific examples of the inventive compound per glycerin concentrations include but are not limited to 2, 5, 10, 20, 22, 25, 40 and 50 mg/ml.

Different grades of glycerin (synonyms: 1,2,3-propanetriol; glycerol; glycol alcohol; glycerol anhydrous) may be used to prepare the formulations. Preferably, glycerin with chemical purity higher than 90% is used to prepare the formulations.

In another embodiment, decitabine or azacitidine is dissolved in propylene glycol at different concentrations. For example, the formulation may optionally comprise between 0.1 and 200; between 0.1 and 100; between 0.1 and 50; between 2 and 50; between 2 and 100; between 5 and 100; between 10 and 100 or between 20 and 100 mg inventive compound per ml of propylene glycol. Specific examples of decitabine per propylene glycol concentrations include but are not limited to 2, 5, 10, 20, 22, 40 and 50 mg/ml.

In yet another embodiment, decitabine or azacitidine is dissolved in a solvent combining glycerin and propylene glycol at different concentrations. The concentration of propylene glycol in the solvent is between 0.1-99.9, optionally between 1-90%, between 10-80%, or between 50-70%.

In yet another embodiment, decitabine or azacitidine is dissolved at different concentrations in a solvent combining glycerin and polyethylene glycol (PEG) such as PEG300, PEG400 and PEG1000. The concentration of polyethylene glycol in the solvent is between 0.1-99.9%, optionally between 1-90%, between 10-80%, or between 50-70%.

In yet another embodiment, the decitabine or azacitidine is dissolved at different concentrations in a solvent combining propylene glycol, polyethylene glycol and glycerin. The concentration of propylene glycol in the solvent is between 0.1-99.9%, optionally between 1-90%, between 10-60%, or between 20-40%; and the concentration of polyethylene glycol in the solvent is between 0.1-99.9%, optionally between 1-90%, between 10-80%, or between 50-70%.

It is believed and experimentally proven that addition of propylene glycol can further improve chemical stability, reduce viscosity of the formulations and facilitate dissolution of decitabine or azacitidine in the solvent.

The pharmaceutical formulation may further comprise an acidifying agent added to the formulation in a proportion such that the formulation has a resulting pH between about 4 and 8. The acidifying agent may be an organic acid. Examples of organic acid include, but are not limited to, ascorbic acid, citric acid, tartaric acid, lactic acid, oxalic acid, formic acid, benzene sulphonic acid, benzoic acid, maleic acid, glutamic acid, succinic acid, aspartic acid, diatrizoic acid, and acetic acid. The acidifying agent may also be an inorganic acid, such as hydrochloric acid, sulphuric acid, phosphoric acid, and nitric acid.

It is believed that adding an acidifying agent to the formulation to maintain a relatively neutral pH (e.g., within pH 4-8) facilitates ready dissolution of the inventive compound in the solvent and enhances long-term stability of the formulation. In alkaline solution, there is a rapid reversible decomposition of decitabine to N-(formylamidino)-N′-β-D-2-deoxyribofuranosylurea, which decomposes irreversibly to form 1-β-D-2′-deoxyribofuranosyl-3-guanylurea. The first stage of the hydrolytic degradation involves the formation of N-amidinium-N′-(2-deoxy-β-D-erythropentofuranosyl)urea formate (AUF). The second phase of the degradation at an elevated temperature involves formation of guanidine. In acidic solution, N-(formylamidino)-N′-β-D-2-deoxyribofuranosylurea and some unidentified compounds are formed. In strongly acidic solution (at pH<2.2) 5-azacytosine is produced. Thus, maintaining a relative neutral pH may be advantageous for the formulation comprising decitabine or azacitidine.

In a variation, the acidifying agent is ascorbic acid at a concentration of 0.01-0.2 mg/ml of the solvent, optionally 0.04-0.1 mg/ml or 0.03-0.07 mg/ml of the solvent.

The pH of the pharmaceutical formulation may be adjusted to be between pH 4 and pH 8, preferably between pH 5 and pH 7, and more preferably between pH 5.5 and pH 6.8.

The pharmaceutical formulation is preferably at least 80%, 90%, 95% or more stable upon storage at 25° C. for 7, 14, 21, 28 or more days. The pharmaceutical formulation is also preferably at least 80%, 90%, 95% or more stable upon storage at 40° C. for 7, 14, 21, 28 or more days.

In one embodiment, the pharmaceutical formulation is prepared by taking glycerin and dissolving the inventive compound in the glycerin. This may be done, for example, by adding decitabine or azacitidine to the glycerin or by adding the glycerin to decitabine. By their admixture, the pharmaceutical formulation is formed.

Optionally, the method further comprises additional steps to increase the rate at which decitabine or azacitidine is solvated by the glycerin. Examples of additional steps that may be performed include, but are nor limited to, agitation, heating, extension of solvation period, and application of micronized inventive compound and the combinations thereof.

In one variation, agitation is applied. Examples of agitation include but are nor limited to, mechanical agitation, sonication, conventional mixing, conventional stirring and the combinations thereof. For example, mechanical agitation of the formulations may be performed according to manufacturer's protocols by Silverson homogenizer manufactured by Silverson Machines Inc., (East Longmeadow, Mass.).

In another variation, heat may be applied. Optionally, the formulations may be heated in a water bath. Preferably, the temperature of the heated formulations may be less than 70° C., more preferably, between 25° C. and 40° C. As an example, the formulation may be heated to 37° C.

In yet another variation, decitabine or azacitidine is solvated in glycerin over an extended period of time.

In yet another variation, a micronized form of the inventive compound may also be employed to enhance solvation kinetics. Optionally, micronization may be performed by a milling process. As an example, micronization may be performed by milling process performed Mastersizer using an Air Jet Mill, manufactured by IncFluid Energy Aljet Inc. (Boise, Id. Telford, Pa.).

Optionally, the method further comprises adjusting the pH of the pharmaceutical formulations by commonly used methods. In one variation, pH is adjusted by addition of acid, such as ascorbic acid, or base, such as sodium hydroxide. In another variation, pH is adjusted and stabilized by addition of buffered solutions, such as solution of (Ethylenedinitrilo) tetraacetic acid disodium salt (EDTA). As decitabine is known to be pH-sensitive, adjusting the pH of the pharmaceutical formulations to approximately pH 7 may increase the stability of therapeutic component.

Optionally, the method further comprises separation of non-dissolved decitabine or azacitidine from the pharmaceutical formulations. Separation may be performed by any suitable technique. For example, a suitable separation method may include one or more of filtration, sedimentation, and centrifugation of the pharmaceutical formulations. Clogging that may be caused by non-dissolved particles of the inventive compound, may become an obstacle for administration of the pharmaceutical formulations and a potential hazard for the patient. The separation of non-dissolved decitabine or azacitidine from the pharmaceutical formulations may facilitate administration and enhance safety of the therapeutic product.

Optionally, the method further comprises sterilization of the pharmaceutical formulations. Sterilization may be performed by any suitable technique. For example, a suitable sterilization method may include one or more of sterile filtration, chemical, irradiation, heat, and addition of a chemical disinfectant to the pharmaceutical formulation.

As noted, decitabine is unstable in water and hence it may be desirable to reduce the water content of the glycerin used for formulating decitabine or azacitidine. Accordingly, prior to the dissolution and/or sterilization step, the glycerin may be dried. Such drying of glycerin or the solution of the inventive compound in glycerin may be achieved by the addition of a pharmaceutically acceptable drying agent to the glycerin. The glycerin or the inventive formulations may be dried, for example by filtering it through a layer comprising a drying agent.

Optionally, the method may further comprise adding one or more members of the group selected from drying agents, buffering agents, antioxidants, stabilizers, antimicrobials, and pharmaceutically inactive agents. In one variation, antioxidants such as ascorbic acid, ascorbate salts and mixtures thereof may be added. In another variation stabilizers like glycols may be added.

3. Vessels or Kits

The pharmaceutical formulations, described in this invention, may be contained in a sterilized vessel such as syringes, vials or ampoules of various sizes and capacities. The sterilized vessel may optionally contain between 1-50 ml, 1-25 ml or 1-20 ml or 1-10 ml of the formulations. Sterilized vessels maintain sterility of the pharmaceutical formulations, facilitate transportation and storage, and allow administration of the pharmaceutical formulations without prior sterilization step.

The present invention also provides a kit for administering the DNA methylation inhibitor (e.g., decitabine and azacitidine) to a host in need thereof. In one embodiment, the kit comprises decitabine or azacitidine in a solid, preferably powder form, and a non-aqueous diluent that comprises glyercin, propylene glycol, polyethylene glycol, or combinations thereof. Mixing of the solid decitabine and the diluent preferably results in the formation of a pharmaceutical formulation according to the present invention. For example, the kit may comprise a first vessel comprising decitabine or azacitidine in a solid form; and a vessel container comprising a diluent that comprises glyercin; wherein adding the diluent to solid decitabine or azacitidine results in the formation of a pharmaceutical formulation for administering the inventive compound. Mixing solid decitabine or azacitidine and diluent may optionally form a pharmaceutical formulation that comprises between 0.1 and 200 mg of the inventive compound per ml of the diluent, optionally between 0.1 and 100, between 2 mg and 50 mg, 5 mg and 30 mg, between 10 mg and 25 mg per ml of the solvent.

According to the embodiment, the diluent is a combination of propylene glycol and glycerin, wherein the concentration of propylene glycol in the solvent is between 0.1-99.9%, optionally between 1-90%, between 10-60%, or between 20-40%.

Also according to the embodiment, the diluent is a combination of polyethylene glycol and glycerin, wherein the concentration of polyethylene glycol in the solvent is between 0.1-99.9%, optionally between 1-90%, between 10-60%, or between 20-40%.

Also according to the embodiment, the diluent is a combination of propylene glycol, polyethylene glycol and glycerin, wherein the concentration of propylene glycol in the solvent is between 0.1-99.9%, optionally between 1-90%, between 10-60%, or between 20-40%; and the concentration of polyethylene glycol in the solvent is between 0.1-99.9%, optionally between 1-90%, between 10-60%, or between 20-40%.

The diluent also optionally comprises 40%, 20%, 10%, 5%, 2% or less water. In one variation, the diluent is anhydrous and may optionally further comprise a drying agent. The diluent may also optionally comprise one or more drying agents, glycols, antioxidants and/or antimicrobials.

The kit may optionally further include instructions. The instructions may describe how solid decitabine or azacitidine and the diluent should be mixed to form a pharmaceutical formulation. The instructions may also describe how to administer the resulting pharmaceutical formulation to a patient. It is noted that the instructions may optionally describe the administration methods according to the present invention.

The diluent and the DNA methylation inhibitor may be contained in separate vessels. The vessels may come in different sizes. For example, the vessel may comprise between 1 and 50, 1 and 25, 1 and 20, or 1 and 10 ml of the diluent.

The pharmaceutical formulations provided in vessels or kits may be in a form that is suitable for direct administration or may be in a concentrated form that requires dilution relative to what is administered to the patient. For example, pharmaceutical formulations, described in this invention, may be in a form that is suitable for direct administration via infusion.

The methods and kits described herein provide flexibility wherein stability and therapeutic effect of the pharmaceutical formulations comprising the DNA methylation inhibitor may be further enhanced or complemented.

4. Routes of Administration

The compounds or formulations in the present invention can be administered by any route, preferably in the form of a pharmaceutical composition adapted to such a route, as illustrated below and are dependent on the condition being treated. The compounds or formulations can be, for example, administered orally, parenterally, topically, intraperitoneally, intravenously, intraarterially, transdermally, sublingually, intramuscularly, rectally, transbuccally, intranasally, liposomally, via inhalation, vaginally, intraoccularly, via local delivery (for example by catheter or stent), subcutaneously, intraadiposally, intraarticularly, or intrathecally. The compounds and/or compositions according to the invention may also be administered or co-administered in slow release dosage forms.

The compounds and/or compositions of this invention may be administered or co-administered in any conventional dosage form. Co-administration in the context of this invention is defined to mean the administration of more than one therapeutic agent in the course of a coordinated treatment to achieve an improved clinical outcome. Such co-administration may also be coextensive, that is, occurring during overlapping periods of time.

The pharmaceutical formulations may be co-administered in any conventional form with one or more member selected from the group comprising infusion fluids, therapeutic compounds, nutritious fluids, anti-microbial fluids, buffering and stabilizing agents.

As described above, the DNA methylation inhibitor can be formulated in a liquid form by solvating the inventive compound in an aqueous solution such as WFI or a non-aqueous solvent such as glycerin. The pharmaceutical liquid formulations provide the further advantage of being directly administrable, (e.g., without further dilution) and thus can be stored in a stable form until administration. Further, because glycerin can be readily mixed with water, the formulations can be easily and readily further diluted just prior to administration. For example, the pharmaceutical formulations can be diluted with water 180, 60, 40, 30, 20, 10, 5, 2, 1 minute or less before administration to a patient.

Patients may receive the pharmaceutical formulations intravenously. The preferred route of administration is by intravenous infusion. Optionally, the pharmaceutical formulations of the current invention may be infused directly, without prior reconstitution.

In one embodiment, the pharmaceutical formulation is infused through a connector, such as a Y site connector, that has three arms, each connected to a tube. As an example, Baxter® Y-connectors of various sizes can be used. A vessel containing the pharmaceutical formulation is attached to a tube further attached to one arm of the connector. Infusion fluids, such as 0.9% sodium chloride, or 5% dextrose, or 5% glucose, or Lactated Ringer's, are infused through a tube attached to the other arm of the Y-site connector. The infusion fluids and the pharmaceutical formulations are mixed inside the Y site connector. The resulting mixture is infused into the patient through a tube connected to the third arm of the Y site connector. The advantage of this administration approach over the prior art is that the inventive compound is mixed with infusion fluids before it enters the patient's body, thus reducing the time when decomposition of the DNA methylation inhibitor such as decitabine may occur due to contact with water. For example, the formulation of decitabine is mixed less than 10, 5, 2 or 1 minutes before entering the patient's body.

Patients may be infused with the pharmaceutical formulations for 1, 2, 3, 4, 5 or more hours, as a result of the enhanced stability of the formulations in a nonaqueous solution. Prolonged periods of infusion enable flexible schedules of administration of therapeutic formulations.

Alternatively or in addition, speed and volume of the infusion can be regulated according to the patient's needs. The regulation of the infusion of the pharmaceutical formulations can be performed according to existing protocols.

The pharmaceutical formulations may be co-infused in any conventional form with one or more member selected from the group comprising infusion fluids, therapeutic compounds, nutritious fluids, anti-microbial fluids, buffering and stabilizing agents. Optionally, therapeutic components include, but are not limited to, anti-neoplastic agents, alkylating agents, agents that are members of the retinoids superfamily, antibiotic agents, hormonal agents, plant-derived agents, biologic agents, interleukins, interferons, cytokines, immuno-modulating agents, and monoclonal antibodies, may be co-infused with the inventive formulations.

5. Combination Therapy

The DNA methylation inhibitor may be used in conjunction with other therapeutic components including but not limiting to bone marrow or hemapoietic stem cell transplants, anti-neoplastic agents, alkylating agents, agents that are members of the retinoids superfamily, antibiotic agents, hormonal agents, plant-derived agents, biologic agents, interleukins, interferons, cytokines, immuno-modulating agents, and monoclonal antibodies.

In one embodiment, an alkylating agent is used in combination with the DNA methylation inhibitor. Examples of alkylating agents include, but are not limited to bischloroethylamines (nitrogen mustards, e.g. chlorambucil, cyclophosphamide, ifosfamide, mechlorethamine, melphalan, uracil mustard), aziridines (e.g. thiotepa), alkyl alkone sulfonates (e.g. busulfan), nitrosoureas (e.g. carmustine, lomustine, streptozocin), nonclassic alkylating agents (altretamine, dacarbazine, and procarbazine), platinum compounds (carboplastin and cisplatin).

In another embodiment, cisplatin, carboplatin or cyclophosphamide is used in combination with and/or added to the inventive compound/formulation.

In another embodiment, a member of the retinoids superfamily is used in combination with the DNA methylation inhibitor. Retinoids are a family of structurally and functionally related molecules that are derived or related to vitamin A (all-trans-retinol). Examples of retinoid include, but are not limited to, all-trans-retinol, all-trans-retinoic acid (tretinoin), 13-cis retinoic acid (isotretinoin) and 9-cis-retinoic acid.

In yet another embodiment, a hormonal agent is used in combination with the DNA methylation inhibitor. Examples of such a hormonal agent are synthetic estrogens (e.g. diethylstibestrol), antiestrogens (e.g. tamoxifen, toremifene, fluoxymesterol and raloxifene), antiandrogens (bicalutamide, nilutamide, flutamide), aromatase inhibitors (e.g., aminoglutethimide, anastrozole and tetrazole), ketoconazole, goserelin acetate, leuprolide, megestrol acetate and mifepristone.

In yet another embodiment, a plant-derived agent is used in combination with the DNA methylation inhibitor. Examples of plant-derived agents include, but are not limited to, vinca alkaloids (e.g., vincristine, vinblastine, vindesine, vinzolidine and vinorelbine), camptothecin (20(S)-camptothecin, 9-nitro-20(S)-camptothecin, and 9-amino-20(S)-camptothecin), podophyllotoxins (e.g., etoposide (VP-16) and teniposide (VM-26)), and taxanes (e.g., paclitaxel and docetaxel).

In yet another embodiment, a biologic agent is used in combination with the DNA methylation inhibitor, such as immuno-modulating proteins such as cytokines, monoclonal antibodies against tumor antigens, tumor suppressor genes, and cancer vaccines.

Examples of interleukins that may be used in combination with the DNA methylation inhibitor include, but are not limited to, interleukin 2 (IL-2), and interleukin 4 (IL-4), interleukin 12 (IL-12). Examples of interferons that may be used in conjunction with the DNA methylation inhibitor include, but are not limited to, interferon α, interferon β (fibroblast interferon) and interferon γ (fibroblast interferon). Examples of such cytokines include, but are not limited to erythropoietin (epoietin α), granulocyte-CSF (filgrastim), and granulocyte, macrophage-CSF (sargramostim). Immuno-modulating agents other than cytokines include, but are not limited to bacillus Calmette-Guerin, levamisole, and octreotide.

Example of monoclonal antibodies against tumor antigens that can be used in conjunction with the DNA methylation inhibitor include, but are not limited to, HERCEPTIN® (Trastruzumab), RITUXAN® (Rituximab), MYLOTARG® (anti-CD33), and CAMPATH® (anti-CD52).

6. Indications

The therapy according to the present invention may be used to treat a wide variety of diseases that are sensitive to the treatment with a DNA methylation inhibitor such as decitabine and azacitidine, especially epigenetic diseases associated with aberrant DNA methylation.

Preferable indications include benign tumors, various types of cancers such as primary tumors and tumor metastasis, restenosis (e.g. coronary, carotid, and cerebral lesions), hematological disorders or malignancy, abnormal stimulation of endothelial cells (atherosclerosis), insults to body tissue due to surgery, abnormal wound healing, abnormal angiogenesis, diseases that produce fibrosis of tissue, repetitive motion disorders, disorders of tissues that are not highly vascularized, and proliferative responses associated with organ transplants.

Generally, cells in a benign tumor retain their differentiated features and do not divide in a completely uncontrolled manner. A benign tumor is usually localized and nonmetastatic. Specific types benign tumors that can be treated using the present invention include hemangiomas, hepatocellular adenoma, cavernous haemangioma, focal nodular hyperplasia, acoustic neuromas, neurofibroma, bile duct adenoma, bile duct cystanoma, fibroma, lipomas, leiomyomas, mesotheliomas, teratomas, myxomas, nodular regenerative hyperplasia, trachomas and pyogenic granulomas.

In a malignant tumor cells become undifferentiated, do not respond to the body's growth control signals, and multiply in an uncontrolled manner. The malignant tumor is invasive and capable of spreading to distant sites (metastasizing). Malignant tumors are generally divided into two categories: primary and secondary. Primary tumors arise directly from the tissue in which they are found. A secondary tumor, or metastasis, is a tumor which is originated elsewhere in the body but has now spread to a distant organ. The common routes for metastasis are direct growth into adjacent structures, spread through the vascular or lymphatic systems, and tracking along tissue planes and body spaces (peritoneal fluid, cerebrospinal fluid, etc.)

Specific types of cancers or malignant tumors, either primary or secondary, that can be treated using this invention include breast cancer, skin cancer, bone cancer, prostate cancer, liver cancer, lung cancer, brain cancer, cancer of the larynx, gall bladder, pancreas, rectum, parathyroid, thyroid, adrenal, neural tissue, head and neck, colon, stomach, bronchi, kidneys, basal cell carcinoma, squamous cell carcinoma of both ulcerating and papillary type, metastatic skin carcinoma, osteo sarcoma, Ewing's sarcoma, veticulum cell sarcoma, myeloma, giant cell tumor, small-cell lung tumor, gallstones, islet cell tumor, primary brain tumor, acute and chronic lymphocytic and granulocytic tumors, hairy-cell tumor, adenoma, hyperplasia, medullary carcinoma, pheochromocytoma, mucosal neuronms, intestinal ganglloneuromas, hyperplastic corneal nerve tumor, marfanoid habitus tumor, Wilm's tumor, seminoma, ovarian tumor, leiomyomater tumor, cervical dysplasia and in situ carcinoma, neuroblastoma, retinoblastoma, soft tissue sarcoma, malignant carcinoid, topical skin lesion, mycosis fungoide, rhabdomyosarcoma, Kaposi's sarcoma, osteogenic and other sarcoma, malignant hypercalcemia, renal cell tumor, polycythermia vera, adenocarcinoma, glioblastoma multiforma, leukemias, lymphomas, malignant melanomas, epidermoid carcinomas, and other carcinomas and sarcomas.

Hematologic disorders include abnormal growth of blood cells which can lead to dysplastic changes in blood cells and hematologic malignancies such as various leukemias. Examples of hematologic disorders include but are not limited to acute myeloid leukemia, acute promyelocytic leukemia, acute lymphoblastic leukemia, chronic myelogenous leukemia, the myelodysplastic syndromes, thalassemia and sickle cell anemia.

Acute myeloid leukemia (AML) is the most common type of acute leukemia that occurs in adults. Several inherited genetic disorders and immunodeficiency states are associated with an increased risk of AML. These include disorders with defects in DNA stability, leading to random chromosomal breakage, such as Bloom's syndrome, Fanconi's anemia, Li-Fraumeni kindreds, ataxia-telangiectasia, and X-linked agammaglobulinemia.

Acute promyelocytic leukemia (APML) represents a distinct subgroup of AML. This subtype is characterized by promyelocytic blasts containing the 15;17 chromosomal translocation. This translocation leads to the generation of the fusion transcript comprised of the retinoic acid receptor and a sequence PML.

Acute lymphoblastic leukemia (ALL) is a heterogenerous disease with distinct clinical features displayed by various subtypes. Reoccurring cytogenetic abnormalities have been demonstrated in ALL. The most common cytogenetic abnormality is the 9;22 translocation. The resultant Philadelphia chromosome represents poor prognosis of the patient.

Chronic myelogenous leukemia (CML) is a clonal myeloproliferative disorder of a pluripotent stem cell. CML is characterized by a specific chromosomal abnormality involving the translocation of chromosomes 9 and 22, creating the Philadelphia chromosome. Ionizing radiation is associated with the development of CML.

The myelodysplastic syndromes (MDS) are heterogeneous clonal hematopoietic stem cell disorders grouped together because of the presence of dysplastic changes in one or more of the hematopoietic lineages including dysplastic changes in the myeloid, erythroid, and megakaryocytic series. These changes result in cytopenias in one or more of the three lineages. Patients afflicted with MDS typically develop complications related to anemia, neutropenia (infections), or thrombocytopenia (bleeding). Generally, from about 10% to about 70% of patients with MDS develop acute leukemia.

Treatment of abnormal cell proliferation due to insults to body tissue during surgery may be possible for a variety of surgical procedures, including joint surgery, bowel surgery, and cheloid scarring. Diseases that produce fibrotic tissue include emphysema. Repetitive motion disorders that may be treated using the present invention include carpal tunnel syndrome. An example of cell proliferative disorders that may be treated using the invention is a bone tumor.

The proliferative responses associated with organ transplantation that may be treated using this invention include those proliferative responses contributing to potential organ rejections or associated complications. Specifically, these proliferative responses may occur during transplantation of the heart, lung, liver, kidney, and other body organs or organ systems.

Abnormal angiogenesis that may be may be treated using this invention include those abnormal angiogenesis accompanying rheumatoid arthritis, ischemic-reperfusion related brain edema and injury, cortical ischemia, ovarian hyperplasia and hypervascularity, (polycystic ovary syndrome), endometriosis, psoriasis, diabetic retinopaphy, and other ocular angiogenic diseases such as retinopathy of prematurity (retrolental fibroplastic), muscular degeneration, corneal graft rejection, neuromuscular glaucoma and Oster Webber syndrome.

Diseases associated with abnormal angiogenesis require or induce vascular growth. For example, corneal angiogenesis involves three phases: a pre-vascular latent period, active neovascularization, and vascular maturation and regression. The identity and mechanism of various angiogenic factors, including elements of the inflammatory response, such as leukocytes, platelets, cytokines, and eicosanoids, or unidentified plasma constituents have yet to be revealed.

In another embodiment, the pharmaceutical formulations of the present invention may be used for treating diseases associated with undesired or abnormal angiogenesis. The method comprises administering to a patient suffering from undesired or abnormal angiogenesis the pharmaceutical formulations of the present invention alone, or in combination with anti-neoplastic agent whose activity as an anti-neoplastic agent in vivo is adversely affected by high levels of DNA methylation. The particular dosage of these agents required to inhibit angiogenesis and/or angiogenic diseases may depend on the severity of the condition, the route of administration, and related factors that can be decided by the attending physician. Generally, accepted and effective daily doses are the amount sufficient to effectively inhibit angiogenesis and/or angiogenic diseases.

According to this embodiment, the pharmaceutical formulations of the present invention may be used to treat a variety of diseases associated with undesirable angiogenesis such as retinal/choroidal neuvascularization and corneal neovascularization. Examples of retinal/choroidal neuvascularization include, but are not limited to, Bests diseases, myopia, optic pits, Stargarts diseases, Pagets disease, vein occlusion, artery occlusion, sickle cell anemia, sarcoid, syphilis, pseudoxanthoma elasticum carotid abostructive diseases, chronic uveitis/vitritis, mycobacterial infections, Lyme's disease, systemic lupus erythematosis, retinopathy of prematurity, Eales disease, diabetic retinopathy, macular degeneration, Bechets diseases, infections causing a retinitis or chroiditis, presumed ocular histoplasmosis, pars planitis, chronic retinal detachment, hyperviscosity syndromes, toxoplasmosis, trauma and post-laser complications, diseases associated with rubesis (neovascularization of the angle) and diseases caused by the abnormal proliferation of fibrovascular or fibrous tissue including all forms of proliferative vitreoretinopathy. Examples of corneal neuvascularization include, but are not limited to, epidemic keratoconjunctivitis, Vitamin A deficiency, contact lens overwear, atopic keratitis, superior limbic keratitis, pterygium keratitis sicca, sjogrens, acne rosacea, phylectenulosis, diabetic retinopathy, retinopathy of prematurity, corneal graft rejection, Mooren ulcer, Terrien's marginal degeneration, marginal keratolysis, polyarteritis, Wegener sarcoidosis, Scleritis, periphigoid radial keratotomy, neovascular glaucoma and retrolental fibroplasia, syphilis, Mycobacteria infections, lipid degeneration, chemical burns, bacterial ulcers, fungal ulcers, Herpes simplex infections, Herpes zoster infections, protozoan infections and Kaposi sarcoma.

In yet another embodiment, the pharmaceutical formulations of the present invention may be used for treating chronic inflammatory diseases associated with abnormal angiogenesis. The method comprises administering to a patient suffering from a chronic inflammatory disease associated with abnormal angiogenesis the pharmaceutical formulations of the present invention alone, or in combination with an anti-neoplastic agent whose activity as an anti-neoplastic agent in vivo is adversely affected by high levels of DNA methylation. The chronic inflammation depends on continuous formation of capillary sprouts to maintain an influx of inflammatory cells. The influx and presence of the inflammatory cells produce granulomas and thus, maintains the chronic inflammatory state. Inhibition of angiogenesis using the pharmaceutical formulations of the present invention may prevent the formation of the granulosmas, thereby alleviating the disease. Examples of chronic inflammatory disease include, but are not limited to, inflammatory bowel diseases such as Crohn's disease and ulcerative colitis, psoriasis, sarcoidois, and rheumatoid arthritis.

Inflammatory bowel diseases such as Crohn's disease and ulcerative colitis are characterized by chronic inflammation and angiogenesis at various sites in the gastrointestinal tract. For example, Crohn's disease occurs as a chronic transmural inflammatory disease that most commonly affects the distal ileum and colon but may also occur in any part of the gastrointestinal tract from the mouth to the anus and perianal area. Patients with Crohn's disease generally have chronic diarrhea associated with abdominal pain, fever, anorexia, weight loss and abdominal swelling. Ulcerative colitis is also a chronic, nonspecific, inflammatory and ulcerative disease arising in the colonic mucosa and is characterized by the presence of bloody diarrhea. These inflammatory bowel diseases are generally caused by chronic granulomatous inflammation throughout the gastrointestinal tract, involving new capillary sprouts surrounded by a cylinder of inflammatory cells. Inhibition of angiogenesis by the pharmaceutical formulations of the present invention should inhibit the formation of the sprouts and prevent the formation of granulomas. The inflammatory bowel diseases also exhibit extra intestinal manifectations, such as skin lesions. Such lesions are characterized by inflammation and angiogenesis and can occur at many sites other the gastrointestinal tract. Inhibition of angiogenesis by the pharmaceutical formulations of the present invention should reduce the influx of inflammatory cells and prevent the lesion formation.

Sarcoidois, another chronic inflammatory disease, is characterized as a multi-system granulomatous disorder. The granulomas of this disease can form anywhere in the body and, thus, the symptoms depend on the site of the granulomas and whether the disease is active. The granulomas are created by the angiogenic capillary sprouts providing a constant supply of inflammatory cells. By using the pharmaceutical formulations of the present invention to inhibit angionesis, such granulomas formation can be inhibited. Psoriasis, also a chronic and recurrent inflammatory disease, is characterized by papules and plaques of various sizes. Treatment using the pharmaceutical formulations of the present invention should prevent the formation of new blood vessels necessary to maintain the characteristic lesions and provide the patient relief from the symptoms.

Rheumatoid arthritis (RA) is also a chronic inflammatory disease characterized by non-specific inflammation of the peripheral joints. It is believed that the blood vessels in the synovial lining of the joints undergo angiogenesis. In addition to forming new vascular networks, the endothelial cells release factors and reactive oxygen species that lead to pannus growth and cartilage destruction. The factors involved in angiogenesis may actively contribute to, and help maintain, the chronically inflamed state of rheumatoid arthritis. Treatment using the pharmaceutical formulations of the present invention alone or in conjunction with other anti-RA agents may prevent the formation of new blood vessels necessary to maintain the chronic inflammation and provide the RA patient relief from the symptoms.

In yet another embodiment, the pharmaceutical formulations of the present invention may be used for treating diseases associated with abnormal hemoglobin synthesis. The method comprises administering the pharmaceutical formulations of the present invention to a patient suffering from disease associated with abnormal hemoglobin synthesis. Decitabine containing formulations stimulate fetal hemoglobin synthesis because the mechanism of incorporation into DNA is associated with DNA hypomethylation. Examples of diseases associated with abnormal hemoglobin synthesis include, but are not limited to, sickle cell anemia and β-thalassemia.

Although exemplary embodiments of the present invention have been described and depicted, it will be apparent to the artisan of ordinary skill that a number of changes, modifications, or alterations to the invention as described herein may be made, none of which depart from the spirit of the present invention. All such changes, modifications, and alterations should therefore be seen as within the scope of the present invention.

### EXAMPLE

### 1. Chemical Stability of Decitabine in Aqueous Infusion Fluid

In this example, an aqueous formulation of decitabine was prepared. The drug substance is decitabine contained in a vial in a form of lyophilized powder intended for reconstitution. Each vial contains 50 mg of decitabine intended for parenteral administration via continuous infusion. In this embodiment of the invention, the parenteral route of administration is preferred due to its immediate effect and maximum bioavailability. Preclinical studies demonstrated that the presence of cytidine deaminases in the body, particularly in the small intestine, enzymatically degrade decitabine and result in reduced oral bioavailability. Therefore, a parenteral formulation is preferred in order to ensure maximum therapeutic benefit of decitabine. A schedule of 15 mg/m² of drug given every 8 h three times a day (each administration as a continuous infusion) for three consecutive days was used in clinical trials.

A ready-to-use solution formulation of decitabine was not developed because decitabine degrades rapidly in aqueous solution. Maintaining the solution at low temperature and buffering the drug can control degradation kinetics of aqueous solutions but the stability achieved is not sufficient to render the product stable during its entire proposed shelf life. A dry solid formulation of decitabine which is stable was achieved by lyophilization of an aqueous solution. The process of lyophilization to obtain a solid form is considered essential for maximizing the stability of the product.

A bulk solution of 5 mg/mL of decitabine was prepared during compounding. To minimize degradation during compounding of the bulk solution prior to lyophilization, the bulk solution was maintained at low temperature, and the time the drug remains in solution prior to lyophilization was minimized. Maximum solution stability was achieved by dissolving the drug in a pH 6.8-7.0 phosphate buffer solution at 0-4° C.

Decitabine in a form of lyophilized powder offers long-term stability and is eventually reconstituted and diluted in infusion fluids prior to administering to the patient. WFI (water for injection) is the solvent of choice for reconstitution because of ease of solubility of the lyophilized product in water and its compatibility with the standard infusion fluids used in clinical practice. However, because of the aqueous nature of the reconstituted solution and to minimize degradation at higher drug concentration (5 mg/mL) and room temperature it was diluted in infusion fluids in less than fifteen minutes. Unless used immediately the diluted solution is prepared using cold infusion fluids and stored under refrigeration at 2-8° C. until use for a period of time described below.

Stability studies were performed to demonstrate short-term stability of the reconstituted and diluted solutions that simulate pre-administration storage periods that may be used in clinical practice. Based on the results, appropriate recommendations for the preparation and storage of the reconstituted and diluted solutions can be made to pharmacists and clinical practitioners. The product was reconstituted in 10 mL WFI and diluted with one of the three pre-chilled infusion fluids indicated in Table 1 and Table 2 to two different concentrations, 0.1 mg/mL and 1 mg/mL covering the entire concentration range expected to be used in the clinical setting. The diluted solutions were stored for one to ten hours in infusion bags in the refrigerator at 2-8° C. to simulate conditions prior to infusion and then removed and placed at room temperature to simulate conditions during infusion. Bags placed in the refrigerator for one hour were removed and placed at room temperature and sampled thereafter for the next three hours at room temperature (Experiment No. 1) while the bags placed in the refrigerator for ten hours were removed and sampled for the next two hours at room temperature (Experiment No. 2).

All solutions were visually inspected and found to be clear and colorless. The data obtained are tabulated in Table 1 and Table 2, and demonstrate that the drug content remains above 90% in the infusion solution during infusion, provided the infusions are prepared and stored as instructed. The method used to estimate degradation products is based on percent peak area measurements assuming the degradation products have a response factor of 1, with respect to decitabine. Only total impurities are reported in the Tables.

Table 1 and Table 2 also show the pH data obtained after the preparation of the infusion, which is consistently within ±0.3 pH units of neutral pH.

TABLE 1
Decitabine Content and Total Impurities (in Parentheses) in Reconstituted and Diluted Solutions (Drug
Concentration 0.1 mg/mL) of the Drug Product Over 12 Hours

| | | | Decitabine Content (%) | | | | | | | |
|---|---|---|---|---|---|---|---|---|---|---|
| Infusion | | Experiment | (Impurities %) | | | | | | | |
| Fluid | pH | No.* | at 0 h | at 1 h | at 2 h | at 3 h | at 4 h | at 10 h | at 11 h | at 12 h |
| 5% | 6.9 | 1 | 97.4% | 97.2% | 96.4% | 94.9% | 93.6% | | | |
| Dextrose | | | (2.6%) | (2.7%) | (3.6%) | (5.1%) | (6.4%) | | | |
| | | 2 | 98.2% | | | | | 94.9% | 94.5% | 93.3% |
| | | | (1.9%) | | | | | (5.2%) | (5.6%) | (6.7%) |
| 0.9% | 6.7 | 1 | 97.5% | 96.9% | 95.7% | 94.1% | 92.5% | | | |
| NaCl | | | (2.5%) | (3.2%) | (4.3%) | (6.0%) | (7.7%) | | | |
| | | 2 | 97.8% | | | | | 93.4% | 92.3% | 90.8% |
| | | | (2.2%) | | | | | (6.7%) | (7.7%) | (9.2%) |
| Lactated | 6.7 | 1 | 98.3% | 98.1% | 97.0% | 95.9% | 94.9% | | | |
| Ringer's | | | (1.7%) | (1.9%) | (3.0%) | (4.1%) | (5.2%) | | | |
| | | 2 | 98.0% | | | | | 93.4% | 92.6% | 91.8% |
| | | | (2.1%) | | | | | (6.7%) | (7.4%) | (8.3%) |

*Experiment 1: store at 2-8° C. for one hour and then hold at room temperature for testing.
Experiment 2: store at 2-8° C. for ten hours and then hold at room temperature for testing.

TABLE 2
Decitabine Content and Total Impurities (in Parentheses) in Reconstituted and Diluted Solutions (Drug
Concentration of About 1.0 mg/mL) of the Drug Product Over 12 Hours

| | | | Decitabine Content (%) | | | | | | | |
|---|---|---|---|---|---|---|---|---|---|---|
| Infusion | | Experiment | (Impurities %) | | | | | | | |
| Fluid | pH | No.* | at 0 h | at 1 h | at 2 h | at 3 h | at 4 h | at 10 h | at 11 h | at 12 h |
| 5% | 7.1 | 1 | 98.0% | 97.0% | 95.6% | 93.7% | 91.9% | | | |
| Dextrose | | | (2.0%) | (3.1%) | (4.4%) | (6.4%) | (8.1%) | | | |
| | | 2 | 97.7% | | | | | 94.5% | 93.9% | 93.0% |
| | | | (2.4%) | | | | | (5.5%) | (6.1%) | (7.1%) |
| 0.9% | 6.7 | 1 | 98.0% | 96.8% | 94.8% | 92.8% | 91.1% | | | |
| NaCl | | | (2.1%) | (3.3%) | (5.2%) | (7.3%) | (9.0%) | | | |
| | | 2 | 97.4% | | | | | 93.4% | 92.5% | 90.8% |
| | | | (2.7%) | | | | | (6.7%) | (7.6%) | (9.2%) |
| Lactated | 6.8 | 1 | 98.1% | 97.3% | 96.2% | 95.1% | 93.7% | | | |
| Ringer's | | | (2.0%) | (2.7%) | (3.8%) | (4.9%) | (6.4%) | | | |
| | | 2 | 98.0% | | | | | 94.1% | 93.3% | 91.8% |
| | | | (2.1%) | | | | | (5.9%) | (6.8%) | (8.2%) |

*Experiment 1: store at 2-8° C. for one hour and then hold at room temperature for testing.
Experiment 2: store at 2-8° C. for ten hours and then hold at room temperature for testing.

Table 3 summarizes the maximum degradation estimated using a series of infusion schedules (S1 through S7 as denoted in the table) selected to establish degradation limits and acceptable shelf life of the diluted solution. The average degradation rate used for calculations is based on the assumption that degradation in solutions is linear and the slope between the initial and the terminal time point is considered to be worst case. If the solution is prepared appropriately and used immediately, degradation would be minimal. It is recommended that a diluted infusion should not be stored longer than seven hours in a refrigerator for a three hour infusion or longer than 10 hours in a refrigerator for a two hour infusion in order to stay within the 10% maximum limit allowed for degradation.

TABLE 3
Maximum Degradation in the Infusion at Selected Infusion Schedules

| | Maximum |
|---|---|
| | degradation |
| | (% peak area) |
| | 0.1-1 mg/mL |
| Infusion schedule | |
| S1 (6 hr stored @4° C. + 3 hr @ RT during infusion) | 9.72 |
| S2 (7 hr stored @4° C. + 3 hr @ RT during infusion) | 10.12 |
| S3 (7 hr stored @4° C. + 2 hr @ RT during infusion) | 7.68 |
| S4 (8 hr stored @4° C. + 3 hr @ RT during infusion) | 10.52 |
| S5 (8 hr stored @4° C. + 2 hr @ RT during infusion) | 8.08 |
| S6 (9 hr stored @4° C. + 2 hr @ RT during infusion) | 8.48 |
| S7 (10 hr stored @4° C. + 2 hr @ RT during infusion) | 8.88 |
| Average degradation rate %/hr:* | |
| @ 4° C. | 0.40 |
| @ Room Temperature | 2.44 |

*Note: These are estimates only, based on assumptions of linearity.

### 2. Treatment of Patients with Hematological Disorders with Low Dose Decitabine

Patients with intermediate and high-risk MDS and CMML were randomized to one of three decitabine schedules: (1) 20 mg/m² infused intravenously over 1 hour, once daily, for five days; (2) 10 mg/m², infused intravenously over one hour, once daily, for ten days; (3) 10 mg/m², injected subcutaneously twice a day, for five days.

Decitabine was supplied as a lyophilized powder, 50 mg in 20 mL glass vials, for reconstitution with 10 mL sterile water (Supergen, Inc., Dublin Calif., through Pharmachemie B.V., Haarlem, The Netherlands). The reconstituted stock solution contained 5 mg/mL decitabine, 6.8 mg KH₂PO₄, and approximately 1.1 mg/mL NaOH. The reconstituted solution was further diluted for intravenous infusion or subcutaneous injection with pharmaceutically acceptable cold infusion fluids (physiological saline, glucose-saline, lactated Ringer's solution). The time delay between preparation of the infusion/injection solution was kept as short as practicable, with refrigerated storage prior to administration not to exceed 4-5 hours.

Patients received one course of treatment (as defined above) every 28 days. The protocol permitted delays for subsequent cycles for patients whose blood counts showed prolonged myelosuppression, who suffered from severe symptoms secondary to myelosuppression, or who did not recover to within a clinically acceptable range of pre-treatment baseline levels. Patients were allowed 40,000 units of erythropoietin weekly for anemia, or GCSF if needed.

The age of 92 treated patients ranged from 31-90 years (median: 65, with 66% of patients being older than 60); according to IPSS prognostic scores, 25% of patients were classified as intermediate-1, 38% as intermediate-2, 19% as high risk, and 17% as having chronic myelomonocytic leukemia (CMML). Fifty-seven percent of patients showed cytogenetic abnormalities; bone marrow blast percentages exceeding 10% were found in 31% of patients; 17% suffered from secondary MDS. Twenty-seven patients had had prior erythropoietin; 17 had had prior GCSF, and 22 had had other prior therapies.

Following initiation of decitabine treatment, complete remission (CR) was achieved in 32 patients (normalization of the peripheral blood and bone marrow, with no more than 5% bone marrow blasts, a peripheral blood granulocyte count of at least 1.0×10⁹/L, and a platelet count of at least 100×10⁹/L).

Partial remission (PR) was found in seven patients (a peripheral blood granulocyte count of at least 1.0×10⁹/L, and a platelet count of at least 100×10⁹/L; with 6-15% bone marrow blasts or a 50% reduction in pretreatment bone marrow blasts).

Sixteen patients showed a clinical benefit (CB: one or more of the following: (i) increase in platelets by 50% and to above 30×10⁹/L, or: (ii) granulocutes increased by 100% and to above 10⁹/L, or: (iii) hemoglobin increase by at least 2 g/dL transfusion independent, or (iv) splenomegaly reduced by at least 50%, or (v) monocytosis reduced by at least 50% if pretreatment levels were >5×10⁹/L).

Thirteen patients showed a CB (as defined above) combined with bone marrow CR.

Overall, 76% of patients who received at least one course of treatment showed a response. The number of treatment courses in patients who achieved CR ranged from one to six (median: 3). After 55 patients were randomized, the 5-day IV arm was determined statistically superior; therefore, remaining patients were not randomized but received 5-day IV courses of treatment.

Notably, 24/57 patients receiving one or more 5-day IV courses of decitabine (20 mg/m² infused intravenously over 1 hour, once daily, for five days) showed complete remission, compared to 4/14 treated with one or more 5-day subcutaneous courses (10 mg/m², injected subcutaneously twice a day, for five days) and 4/17 treated with one or more 10-day IV courses (10 mg/m², infused intravenously over one hour, once daily, for ten days). There was also more myelosuppression noted in the latter cohort.

These study results demonstrate that decitabine shows favorable and clinically meaningful benefits in MDS when administered either in the form of daily 1-hour infusions or twice-daily subcutaneous injections.

While the present invention is disclosed with reference to preferred embodiments detailed above, it is to be understood that these embodiments are intended in an illustrative or exemplary rather than in a limiting sense, as it is contemplated that modifications will readily occur to those skilled in the art, modifications which will be within the spirit of the invention and the scope of the appended claims. All patents, papers, articles, references and books cited herein are incorporated by reference in their entirety.

What is claimed is:

1. A method for treating a patient with a hematological disorder, comprising:
administering to the patient a therapeutically-effective amount of decitabine at a dose ranging from about 1 to about 50 mg/m² via a 1 hour continuous intravenous infusion per day for 5 consecutive days, which constitutes an initial full treatment cycle.

2. The method according to claim 1, wherein the hematological disorder is selected from the group consisting of acute myeloid leukemia (AML), acute promyelocytic leukemia (APL), acute lymphoblastic leukemia (ALL), chronic myelogenous leukemia (CML), the myelodysplastic syndromes (MDS), thalassemia and sickle cell anemia.

3. The method of claim 2, wherein the dose of decitabine is from about 10 to about 30 mg/m² per day.

4. The method of claim 2, wherein the dose of decitabine is from about 15 to about 25 mg/m² per day.

5. The method of claim 2, wherein the dose of decitabine is about 20 mg/m² per day.

6. The method of claim 2, further comprising:
repeating the full treatment cycle every 4 weeks after the initial administration of decitabine.

7. The method of claim 1, further comprising;
administering to the patient a hematopoietic growth hormone.

8. The method of claim 7, wherein the hematopoietic growth hormone is erythropoietin (EPO), granulocyte colony-stimulating factor (GCSF) or granulocyte macrophage colony-stimulating factor (GMCSF).

9. The method of claim 1, further comprising:
administering an allogenic transplant to the patient.

10. The method of claim 9, wherein the allogenic transplant is bone marrow or hematopoietic stem cells.

11. A method for treating a patient with a hematological disorder, comprising:
administering to the patient a therapeutically-effective amount of decitabine at a dose ranging from about 1 to 50 mg/m² subcutaneously.

12. The method according to claim 11, wherein the hematological disorder is selected from the group consisting of acute myeloid leukemia (AML), acute promyelocytic leukemia (APL), acute lymphoblastic leukemia (ALL), chronic myelogenous leukemia (CML), the myelodysplastic syndromes (MDS), thalassemia and sickle cell anemia.

13. The method of claim 12, wherein the dose of decitabine is from about 10 to about 30 mg/m² per day.

14. The method of claim 12, wherein the dose of decitabine is from about 15 to about 25 mg/m² per day.

15. The method of claim 12, wherein the dose of decitabine is about 20 mg/m² per day.

16. The method of claim 15, wherein decitabine is administered to the patient via bolus injection at a dose of about 10 mg/m² twice a day.

17. The method of claim 12, further comprising: repeating the full treatment cycle every 4 weeks after the initial administration of decitabine.

18. The method of claim 11, further comprising;
administering to the patient a hematopoietic growth hormone.

19. The method of claim 18, wherein the hematopoietic growth hormone is erythropoietin (EPO), granulocyte colony-stimulating factor (GCSF) or granulocyte macrophage colony-stimulating factor (GMCSF).

20. The method of claim 11, further comprising:
administering an allogenic transplant to the patient.

21. The method of claim 20, wherein the allogenic transplant is bone marrow or hematopoietic stem cells.
